(19) United States
(12) Patent Application Publication
(10) Pub. No.: US 2007/0172815 A1
(43) Pub. Date: Jul. 26, 2007

(54) METHOD FOR DETECTING MODULATORS OF ION CHANNELS USING THALLIUM (I) SENSITIVE ASSAYS

(75) Inventor: Charles David Weaver, Franklin, TN (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/696,242

(22) Filed: Apr. 4, 2007

Related U.S. Application Data
(62) Division of application No. 09/975,891, filed on Oct. 12, 2001.
(60) Provisional application No. 60/240,523, filed on Oct. 13, 2000.

(51) Int. Cl. C12Q 1/00; G01N 33/567

(52) U.S. Cl. 435/4; 435/7.2

(57) ABSTRACT

Disclosed and claimed herein are novel thallium-sensitive assays for identifying modulators of ion channels, channel-linked receptors or ion transporters. Further described are novel chloride-free buffers and low chloride cell growth media.

This application is a divisional application of U.S. application Ser. No. 09/975,891, filed Oct. 12, 2001, which claims priority to provisional patent application U.S. Ser. No. 60/240,523, filed Oct. 13, 2000, the contents of both are incorporated by reference in their entirety, into the present application.

Throughout this application various publications are referenced. The disclosures of these publications are hereby incorporated by reference into this application in order to more fully describe the state of the art to which this invention pertains.

### FIELD OF INVENTION

The present invention relates to a method of screening compounds that modulate the activity of ion channels, ion channel linked receptors, or ion transporters using a thallium (I) (T1⁺) sensitive fluorescence assay.

### BACKGROUND OF THE INVENTION

Ion channels are transmembrane proteins that mediate transport of ions across cell membranes. These channels are pervasive throughout most cell types and important for regulating cellular excitability and homeostasis. Ion channels participate in numerous cellular processes such as action potentials, synaptic transmission, hormone secretion, and muscle contraction. Many important biological processes in living cells involve the translocation of cations, such as calcium (Ca²⁺), potassium (K⁺), and sodium (Na⁺) ions, through ion channels. Cation channels represent a large and diverse family of ion channels that are recognized as important drug targets.

Different nomenclature may be used for ion channels. Ion channels can be defined as either ligand- or voltage-gated, selective or non-selective ion channels (North, R.A. 1995, Ligand and Voltage-Gated Ion Channels, CRC Press, Inc.; Boca Raton, Fla., 1-58). For instance, classic voltage-gated potassium channels, sodium channels, and calcium ion channels are generally considered to be selective ion channels because they exhibit strong selectivity or preference for their respective ions under physiological conditions. However, the selectivity is not absolute, as sodium channels can pass other ions, such as lithium. In contrast, non-selective cation channels transport many cations with little or no preference. For example, the alpha-amino-3-hydroxy-5-methyl-4-isoxazoleproprionate (AMPA)-type glutamate receptor ion channel is a ligand-gated non-selective ion channel that will readily pass ions, e.g., lithium, sodium, potassium, rubidium, cesium and calcium ions. Ligand-gated ion channels are regulated by binding of a ligand to the ion channel. Examples of ligand-gated ion channels are glutamate, nicotinic acetylcholine receptors, AMPA, N-methyl-D-aspartate (NMDA) and vanilloid receptors.

Voltage-gated ion channels respond to changes in cell membrane potential by opening or closing the channel, thereby mediating ion transport. These channels are present in excitable (e.g., nerve, muscle) and non-excitable (e.g., exocrine/endocrine secretory, and blood) cells and have crucial roles in cellular signaling and interactions (Conley, E. C.; Brammar, W. J. The Ion Channel FactsBook IV. Voltage-Gated Channels, 1999, Academic Press, London, U.K.) and are therefore important targets of drug discovery. These ion channels have many attributes characteristic of suitable drug targets, for example: (1) they have known biological function; (2) they are modified by and accessible to small molecular weight compounds in vivo; and (3) they have assay systems for in vitro characterization and high-throughput screening (Curran, M. Current Opin. Biotech., 1998, 9, 565-572).

Potassium (K⁺) channels are encoded by a large and diverse gene family of cation channels and are grouped into voltage-gated and ligand-gated subtypes based on their gating properties. These channels are membrane bound macromolecules associated with regulatory functions in nearly all cell types, tissues, and organs (North, R. A. 1995, Ligand and Voltage-Gated Ion Channels, CRC Press, Inc.; Boca Raton, Fla., 1-58). K⁺ channels regulate membrane potential in electrically excitable cells (e.g., nerves and muscle) and in non-excitable cells (e.g., lymphocytes), signal transduction, insulin secretion, hormone release, and vascular tone, cell volume and immune response (Hille, B. Ionic channels of Excitable Membranes, 1992, Ed. 2, Sunderland, Mass.; Sinauer). Recently, K⁺ channels have been identified in important physiologic processes and found to be associated with human diseases including cardiovascular disease, blood pressure/vascular resistance, epilepsy, Sickle cell anemia, skeletal muscle disorders, Islet cell metabolism, immunosuppression, inflammation, and cancer (Bulman, D. E. Hum. Mol. Genet. 1997, 6, 1679-1685; Ackerman, M. J.; Olapham, D. E. N. Engl. J. Med. 1997, 336, 1575-1586; Curran, M., supra).

Voltage-gated K⁺ channels detect changes in membrane potential and respond by transporting K⁺ ions. Ligand-gated K⁺ channels are modulated by small molecular weight effectors, such as calcium, sodium, ATP, or fatty acids (Lazdunski, Cardiovascular Drugs and Therapy, 1992, 6, 313-319). Although, both voltage-gated and ligand-gated K⁺ channels transport potassium ions, they differ in biophysical, biochemical and pharmacological properties. In an attempt to classify potassium ion channels, Doupnik et al., has proposed a systematic nomenclature for the inward rectifying family of K⁺ channel proteins (Doupnik, et al., Curr. Opin. Neuro. 1995, 8, 268-277). The family is characterized by its tertiary structure and a pore region homologous to that of monovalent cation voltage-dependent channels.

The Kir-3 channels are a subfamily of the K⁺ voltage-dependent channel family regulated by G-proteins (Doupnik, et al., supra). G-protein mediated signaling pathways are suggested to be directly coupled to ion channels; i.e., channel-linked receptors. G-protein regulated K⁺ channels, such as G-protein activated inward rectifier K⁺ channels (GIRKs), have been shown to be important for the regulation of heart and nerve function (Kurachi, et al., Prog. Neurobiol., 39, 229-246; Grown, and Birnbaumer, Ann. Rev. Physiol. 1990, 52, 297-213; Mark, M. D. and Herlitze, S., Eur. J. Biochem., 2000, 267, 5830-5836; Leaney, J. L. and Tinker, A.; Proc. Natl. Acad. Sci., 2000, 97, 5651-5656).

The coupling of a neuronal receptor to the atrial K⁺ channel has also been demonstrated by Karschin et al., (Karschin et al., Proc. Nat. Acad. Sci., 1991, 88, 5694-5698). Monitoring the activity of these ion channels, in particular the ion channels linked to the G-protein coupled receptor (GPCR) family of proteins, provides indirect methods for observing the effect of potential modulatory compounds on the activity of the GPCR. As such, GPCRs are notable targets for drug design. Currently, there is a need for facile and efficient high-throughput screening assays to detect compounds that modulate GPCR activity.

Movement of physiologically relevant substrates through ion channels can be traced by a variety of physical, optical, or chemical techniques (Stein, W. D. Transport and Diffusion Across Cell Membranes, 1986, Academic Press, Orlando, Fla.). Assays for modulators of ion channels include electrophysiological assays, cell-by-cell assays using microelectrodes (Wu, C.-F., Suzuki, N., and Poo, M. M. J. Neurosci, 1983, 3 1888) i.e., intracellular and patch clamp techniques (Neher, E.; Sakmann, B., 1992, Sci. Amer., 266, 44-51), and radioactive tracer ion techniques. The patch clamp and whole cell voltage clamp, current clamp, and two-electrode voltage clamp techniques require a high degree of spatial precision when placing the electrodes. Functional assays can be conducted to measure whole-cell currents with the patch clamp technique, however, the throughput is very limited in number of assays per day.

Radiotracer ions have been used for biochemical and pharmacological investigations of channel-controlled ion translocation in cell preparations (Hosford, D. A.; et al., Brain Res., 1990, 516, 192-200). In this method, the cells are exposed to a radioactive tracer ion and an activating ligand for a period of time, the cells are then washed, and counted for radioactive content. Radioactive isotopes are well known (Evans, E. A.; Muramtsu, M. Radiotracer techniques and applications M. Dekker; New York, 1977) and their uses have permitted detection of target substances with high sensitivity. However, radioactive isotopes require many safety precautions. The uses of alternative and safer non-radioactive labeling agents has thus increased in recent years.

Optical methods using fluorescence detection are suitable alternatives to the patch-clamp and radioactive tracer techniques. Optical methods permit measurement of the entire course of ion flux in a single cell as well as in groups of cells. The advantages of monitoring transport by fluorescence techniques include the high level of sensitivity of these methods, temporal resolution, modest demand for biological material, lack of radioactivity, and the ability to continuously monitor ion transport to obtain kinetic information (Eidelman, O. Cabantchik, Z. I. Biochim. Biophys. Acta, 1989, 988, 319-334). The general principle of monitoring transport by fluorescence is based on having compartment-dependent variations in fluorescence properties associated with translocation of compounds.

Optical methods were developed initially for measuring Ca²⁺ ion flux (Scarpa, A. Methods of Enzymology, 1979, 56, 301 Academic Press, Orlando, Fla.; Tsien, R. Y. Biochemistry, 1980, 19, 2396; Grynkiewicz, G., Poenic, M., Tsien, R. Y. J. Biol Chem., 260, 3440) and have been modified for high-throughput assays (U.S. Pat. No. 6,057,114). The flux of Ca²⁺ ion is typically performed using calcium-sensitive fluorescent dyes such as Fluo-3, Fluo-4, Calcium green, and others. (Molecular Probes Inc., Handbook of Fluorescent probes and research chemicals, 7th edition, chapter 1, Eugene, Oreg.). Optical detection of electrical activity in nerve cells is conducted using voltage-sensitive membrane dyes and arrays of photodetectors (Grinvald, A, 1985, Annu. Rev. Neurosci. 8, 263; Loew, L. M., and Simpson, L. L., 1981, Biophys. J. 34, 353; Grinvald, A., et al., 1983, Biophys. J. 39, 301; Grinvald, A., et al., Biophys. J. 42, 195).

Karpen et al., developed an optical method to detect monovalent cation flux in living cells. The method measured ion flux based on fluorescent quenching of an entrapped dye, anthracene-1,5-dicarboxylic acid (ADC), by cesium ion (Cs⁺) in whole cells (Karpen, J. W., Sachs, A. B., Pasquale, E. B., Hess, G. P., Anal. Biochem. 1986, 157, 353-359). This method was used to screen cells that would respond to a particular neurotransmitter. The technique by Karpen et al., can be applied to any system in which Cs⁺ can substitute for Na⁺ or K⁺, and has been shown to be comparable to the tracer ion method. However, most classical K⁺ and sodium channels are highly selective against Cs⁺ and, therefore this method is only useful for non-selective cation channels.

It has been previously reported that thallium ions are transported through a number of K⁺ channels (Hille, B. J Gen. Physiol 1972, 59, 637-58). Thallium fluorescence quenching methods for measuring monovalent cation flux were first developed in reconstituted membrane vesicles (Moore, H-P. H., Raftery, M. A. Proc. Natl, Acad. Sci, 1980, 77, 4509-4513). Thallium was reported to affect the fluorescence of polyanionic fluorescent dye, 8-aminonaphthalene-1,3,6-trisulfonate (ANTS) (Moore, H-P. H., Raftery, supra). This method was further used to resolve ion transport kinetics across membrane vesicles containing purified acetylcholine receptor (Wu, W. C. -S.; Moore, H-P. H.; Raftery, M. A. Proc. Natl, Acad. Sci, 1984, 78, 775-779). Influx of thallium ions into the vesicles was measured by the effect of thallium ions on the fluorescence of the entrapped fluorescent agent, ANTS, (Wu, W, C-S. Moore, H-P. H, Raftery, M. A., supra). However, this method has been limited to using vesicles and was reported not applicable to whole cells, due to the insolubility of thallium chloride under physiological conditions.

Application of the above-described optical or radiotracer methods are limited in their adaptability to high throughput screening methods. For example, high throughput screening methods of Ca²⁺ permeable cation channels are typically performed using calcium-sensitive fluorescent dyes such as Fluo-3, Fluo-4, Calcium green, and others (U.S. Pat. Nos. 6,057,114 and 5,985,214). These screening assays are predominantly applied to channels that pass calcium or other related divalent ions, and thus are largely useless for K⁺ channels. High throughput screens for most other cation channels are performed using voltage-sensitive dyes such as DiBAC (U.S. Pat. No. 5,882,873). These dyes report the changes in transmembrane potential that result from ion flux. However, such methods do not directly distinguish the type of channel carrying the charge that alters membrane potential, and thus are more fraught with artifacts, due to, among other issues, the diversity of ion channels present in a cell, impacting reproducibility.

The limitations of known methods for screening compounds that modulate cation channel activity have hampered the search for novel modulators of cation channels. Moreover, the known assays for channel activity are not amenable to high throughput screening methods which are needed to screen large libraries of potential modulators. Thus, there remains a need in the art for new assay methods for screening and identifying large numbers of candidate compounds that modulate cation channel activity. The present invention fulfills these and other needs.

### SUMMARY OF THE INVENTION

Accordingly, the present invention provides novel thallium sensitive optical assay methods to detect modulators of ion channels, channel-linked receptors or ion transporters. The methods use thallium sensitive assays to measure the functional activity of ion channels, channel-linked receptors or ion transporters in living cells.

The methods of the invention further provide high-throughput screening assays for identifying modulators of ion channels, channel-linked receptors or ion transporters. This provides an assay to screen candidate modulators for their ability to block or activate the activity of ion channels, channel-linked receptors or ion transporters. Using the high-throughput screening assays of the present invention, novel compounds that modulate the activity of ion channels, channel-linked receptors or ion transporters are identified for use in the development of novel therapeutic and diagnostic agents.

The methods of the invention also provide a novel low Chloride (Cl⁻) cell growth medium for growing cells expressing the ion channels, channel-linked receptors or ion transporters of interest, and a novel Cl⁻-free assay buffer for performing the thallium sensitive assays of the invention. In these solutions, thallium ions concentrations greater than 200 mM can be achieved. In one embodiment, the cell growth medium contains less than 2 mM Cl⁻ and the chloride anion is replaced by organic gluconate anion. While it is possible to perform all the assays in known physiological Cl⁻ containing buffers, the novel Cl⁻-free buffer conditions and low Cl⁻ cell growth medium produce more robust and consistent results.

### BRIEF DESCRIPTION OF THE FIGURES

FIG. 1 depicts the thallium influx assay for the Ca²⁺ activated, small conductance K⁺ channel, SK2, as described in Example II, infra. The arrow shows the point where ionomycin and thallium ions were added.

FIG. 2A and B show the thallium influx assay for the Ca²⁺ activated, large conductance K⁺ channel, Maxi-K as described in Example III, infra. A) 100 nM IBTX or B) 15 μM NS-1619. The arrow shows the point where ionomycin, Thallium ions and K⁺ were added.

FIG. 3A and B illustrate thallium influx assay for the voltage-gate K⁺ channel, KCNQ2 as described in Example IV, infra. A) 15 μM DMP-543 or B) 15 μM retigabine. The arrow shows the point where thallium ions or (thallium ions and K⁺) were added.

FIG. 4 shows the thallium influx assay for the ligand-gated non-selective cation channel, VR1 (capsaicin receptor) as described in Example V, infra. The arrow shows the point where capsaicin and thallium ions were added.

FIG. 5 shows the thallium efflux assay for the Ca²⁺ activated, small conductance K³⁰ channel, SK2, as described in Example VI, infra. The arrow shows the point where ionomycin was added.

FIG. 6 shows the Muscarinic acetylcholine receptor assay linked through detection of thallium ions influx through the Ca²⁺ activated, small conductance K⁺ channel, SK2, as described in Example VII, infra. The arrow shows the point where the Muscarinic receptor agonist, oxoteremorine-M (oxo-M), was added.

FIG. 7 depicts a typical experimental protocol for a standard thallium influx assay, as described in Example VIII, infra.

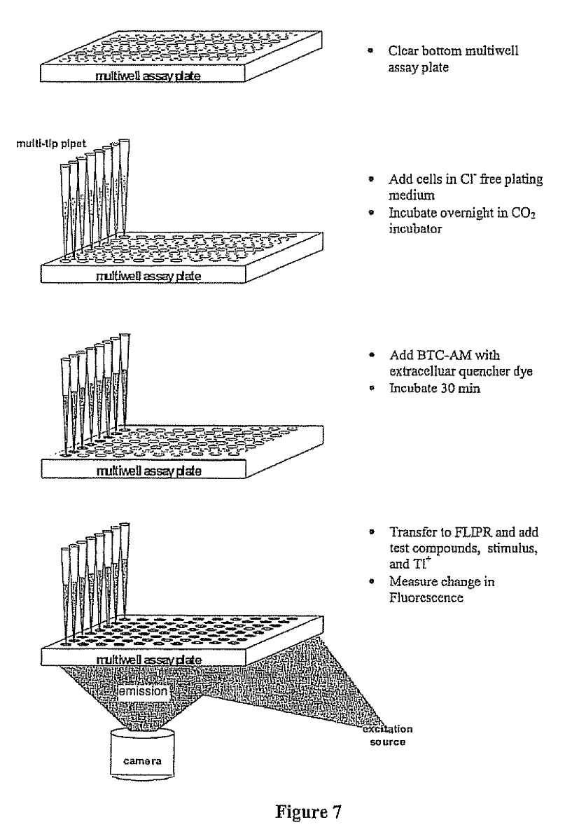

### DETAILED DESCRIPTION OF THE INVENTION

The present invention provides novel thallium-sensitive assay methods using whole cells for detecting and identifying compounds of interest that modulate the activity of ion channels, channel-linked receptors or ion transporters. The compounds of interest either activate or inhibit the activity of the ion channels, channel-linked receptors or ion transporters. Such modulators are valuable research tools that can be used to elucidate the biochemistry, physiology, and pharmacology of ion channels, channel-linked receptors or ion transporters in both prokaryotic and eukaryotic systems.

Moreover, such modulators can provide lead compounds for diagnostic or therapeutic drug development to treat a variety of conditions, including the development of drugs useful for many disorders such as cation channel-associated diseases, diseases associated with channel-linked receptors, antibacterial, antifungal, inflammation modulatory, or immunological disorders.

Accordingly, the assays of the present invention provide methods for identifying lead compounds for pharmaceutical development of drugs that can be used to treat cation channel associated diseases and/or diseases associated with channel-linked receptors. High throughput methods for screening for potential modulators of the activity of ion channels, channel-linked receptors or ion transporters are also provided.

In addition, the methods of the invention include novel low Cl⁻ cell growth medium and Cl⁻-free assay buffer, for conducting the methods of the invention.

Definitions

As used in this application, the following terms or phrases have the meanings specified.

An “ion channel” is any protein or proteins which forms an opening or a pore in a cellular membrane where the pore or opening is capable of permitting ions to flow therethrough.

A “channel-linked receptor” is any protein or proteins which are linked to ion channels, where the protein activity affects the activity of an ion channel.

An “ion transporter” is any protein or proteins which transport ions across a cellular membrane.

A “modulator” is any compound or agent that can alter the activity of an ion channel, i. e., alter the movement or transport of ions through an ion channel. The modulator can be an organic molecule or chemical compound (naturally occurring or non-naturally occurring), such as a biological macromolecule (e.g., nucleic acid, protein, non-peptide, or organic molecule), or an extract made from biological materials such as bacteria, plants, fungi, or animal (particularly mammalian) cells or tissues, protein or protein fragment. Modulators are evaluated for the potential to act as inhibitors or activators of a biological process or processes, e.g., to act as agonist, antagonist, partial agonist, partial antagonist, antineoplastic agents, cytotoxic agents, inhibitors of neoplastic transformation or cell proliferation, and cell proliferation-promoting agents. The activity of a modulator may be known, unknown or partially known.

A channel blocker is a compound that inhibits, directly or indirectly, the movement of ions through an ion channel. The compound may exert its effect by directly occluding the pore, by binding and preventing opening of the pore, or by affecting the time and frequency of the opening of the ion channel.

A channel opener is a compound that activates the movement of ions through an ion channel. The compound may regulate ion channels by effecting the duration and/or frequency of the opening of the ion channel, or change the voltage dependence of voltage-gated ion channels, such that the ion channel is open.

An agonist is a molecule which is able to activate the ion channel, channel-linked receptor or ion transporter.

An antagonist is a molecule which effects the agonist action or which inhibits the activity of the ion channel, channel-linked receptor or ion transporter.

### METHODS OF THE INVENTION

The invention provides novel methods for detecting cation channel modulators using thallium sensitive assays to measure the functional activity of cation channels in living cells. The invention provides for simple and convenient optical methods to detect cation flux (influx or efflux), in particular, the flux of thallium ions. One can measure and observe the activity of the ion channel, directly or indirectly, by detecting the flux of the thallium ions.

In an embodiment of the invention, the method can be used for indirectly detecting modulators of channel-linked receptors that are linked to ion channels. The effect of the modulators of the channel-linked receptors can be observed by measuring and observing the functional activity of the ion channel linked to the receptor (in the presence or absence of the modulator) using the thallium sensitive assays described herein.

In another embodiment of the invention, the method can be used for detecting modulators of ion transporters. The effect of the modulators of the ion transporters can be observed by measuring and observing the functional activity of the ion transporter using the thallium sensitive assays described herein.

General Influx Methods

Methods of the invention include assays for detecting and identifying compounds that are potential modulators of target ion channels, channel-linked receptors or ion transporters using the thallium sensitive assays of the invention. These assays involve incubating a test mixture, that includes cells expressing target ion channels (e.g., potassium ion channel), ion channels that are linked to receptors (e.g., GIRK) and channel-linked receptors (e.g., GPCR), or ion transporters (e.g., glutamate transporter), a detectable (signal generating) thallium sensitive agent (e.g., BTC), thallium ions and a candidate ion channel, channel-linked receptor or ion transporter activity modulator. The optical signal of the thallium sensitive agent is measured before the modulator is added. The assay is performed under conditions that are suitable for the ion channel, channel-linked receptor or ion transporter activity to occur. A change in the optical signal of the thallium sensitive agent is measured. An increase or decrease in the signal indicates the movement of thallium ions through the ion channel or ion transporter.

A method of the invention is practiced using whole cells expressing the ion channels, which includes the steps of: 1) growing cells expressing ion channels under suitable conditions; 2) contacting or loading the cells with a signal generating thallium sensitive agent (e.g., cell permeant thallium sensitive agent); 3) treating the cells under suitable conditions (e.g., washing or adding extracellular quenchers) to remove the contribution of excess thallium sensitive agent outside of the cells; 4) measuring the detectable signal for baseline measurement; 5) contacting the cells with a solution which includes thallium ions and an appropriate stimulus solution (a solution that activates the ion channel, channel-linked receptor, or ion transporter) for the ion channels, contacting the cells with a candidate ion channel modulatory compound; and 6) detecting any signal.

In another embodiment of the invention, the method of the invention is practiced using whole cells expressing ion channels and channel-linked receptors, which includes the steps of: 1) growing cells expressing an ion channel and channel-linked receptor of interest under suitable conditions; 2) contacting or loading the cells with a signal generating thallium sensitive agent (e.g., cell permeant thallium sensitive agent); 3) treating the cells under suitable conditions (e.g., washing or adding extracellular quenchers) to remove excess thallium sensitive agent; 4) measuring the detectable signal for baseline measurement; 5) contacting the cells with a candidate channel-linked receptor modulatory compound; 6) measuring the detectable signal; 7) contacting the cells with a solution which includes thallium ions and an appropriate stimulus solution for the channel-linked receptor; and 8) measuring the detectable signal.

In a further embodiment of the invention, the method of the invention is practiced using whole cells expressing ion transporters, which includes the steps of: 1) growing cells expressing an ion transporter of interest under suitable conditions; 2) contacting or loading the cells with a signal generating thallium sensitive agent (e.g., cell permeant thallium sensitive agent); 3) treating the cells under suitable conditions (e.g., washing or adding extracellular quenchers) to remove excess thallium sensitive agent; 4) measuring the detectable signal for baseline measurement; 5) contacting the cells with a candidate ion transporter modulators; 6) measuring the detectable signal; 7) contacting the cells with a solution which includes thallium ions and an appropriate stimulus solution for the ion transporter; and 8) measuring the detectable signal.

The change in signal generated by the thallium sensitive agent is determined by measuring the baseline signal in the test mixture before the addition of modulator, i.e., before or after the addition of thallium salts or modulator.

A person of ordinary skill in the art will understand that control experiments can be performed to facilitate analysis of the effects of the candidate modulator. Control experiments can be performed using: (1) native, untransfected cells under identical conditions of the methods of the invention; (2) the addition of thallium ions to the test mixture in the absence of stimulus solution; (3) cells under identical conditions of the methods of the invention, but without the candidate modulator of the ion channels, channel-linked receptors or ion transporters added to the test mixture; and/or (4) cells under identical conditions to the methods of the invention, but using known modulators of the ion channels, channel-linked receptors or ion transporters.

General Efflux Method

The present invention further provides methods for measuring the efflux of ions. The methods of measuring thallium influx are described supra and in the example section, infra. The efflux assays use the same cells as in the influx assays, and are loaded with a signal generating thallium sensitive fluorescent agent, as described, such as BTC. The cells are contacted with thallium to load the cells. One embodiment provides contacting the cells with thallium ions for approximately 15 minutes. The cells are washed to remove excess thallium ions and assayed using the same instrument to detect changes in signal as used in the influx assay (e.g., the Fluorometric Inage Plate Reader (FLIPR) (Molecular Devices Corp., Sunnyvale, Calif.)). The assay channels are stimulated to open by the addition of any one of a number of ligands, or by changing the membrane potential of the cell, such as by changing the potassium concentrations, to permit efflux of ions through the ion channels. For BTC, efflux would result in a decrease in fluorescence. The other compounds, such as control compounds, can be the same as used in the influx assay. The same conditions are applied as for the influx assay in the methods of the invention, except the cells are preloaded with thallium ions as described above, and washed to remove excess thallium ions.

Stimulus Solutions

A stimulus solution is a solution that activates the ion channel, channel-linked receptor or ion transporter (e.g., agonist). Some ion channels/transporters may be constitutively active and thus would not require a ‘stimulus’ in addition to the thallium ion tracer. For channels that do require a stimulus, that stimulus may be ligand (a molecule that binds to the channel or channel linked receptor and activates the same (an agonist). A stimulus might also be a change in membrane potential for voltage-gated channels. Typically voltage-gated channels are activated by either direct electrical stimulation with electrodes or by using a stimulus solution that contains an ionic composition that will cause depolarization (such as high external potassium). In addition, thallium ions can also act as a stimulus for voltage-gated channels. In such a case, thallium ions can act as both a ‘tracer’ and a depolarizing stimulus. In an influx assay, thallium ions can be added just before, during, or after the addition of a stimulus.

The methods of the present invention include stimulus solutions that are selected based on the type of ion channel, channel-linked receptor or ion transporter used in the method. Selecting an appropriate stimulus solution and ion channel, channel-linked receptor or ion transporter-activating reagent, is within the skill of the art. In one embodiment, the stimulus solutions include a buffer that does not include reagents that activate the ion channel, such that the ion channels, channel-linked receptors or ion transporters remains substantially at rest. In this embodiment, the stimulus solution includes reagents that do not activate the ion channel, channel-linked receptor or ion transporter of interest but facilitate activation of ion channel, channel-linked receptor or ion transporter when a modulating reagent is added to the cells to initiate the assay.

The stimulus solution selected for use with voltage-dependent ion channels (e.g., the N-type calcium channel or KCNQ2 channel) depends upon the sensitivity of the ion channel to the resting potential of the cell membrane. For methods using these voltage-dependent ion channels, the stimulating solution may include activating reagents that serve to depolarize the membrane (e.g., ionophores, valinomycin, etc.).

A stimulus solution selected for use with some voltage-dependent ion channels for activation by depolarization of the cell membrane includes potassium salt at a concentration such that the final concentration of potassium ions in the cell-containing well is in the range of about 10-150 mM (e.g., 50 mM KCl). In addition, voltage-dependent ion channels may also be stimulated by an electrical stimulus.

The stimulus solution selected for use with channel-linked receptors and ligand-gated ion channels depends upon ligands that are known to activate such receptors. For example, nicotinic acetylcholine receptors are known to be activated by nicotine or acetylcholine; similarly, muscarinic acetyl choline receptors may be activated by addition of muscarine or carbamylcholine. The stimulating solution for use with these systems may include nicotine, acetylcholine, muscarine or carbamylcholine.

Cells

The methods of the invention employ cells having 1) ion channels that are permeable to thallium; 2) ion channels and channel-linked receptors that are permeable to thallium ions; or 3) ion transporters that are permeable to thallium ions. Cells used for the methods of the invention can be generated by transfection of a host cell with DNA encoding an 1) ion channel; 2) ion channel and channel-linked receptor; or 3) ion transporter.

Although essentially any cell which expresses endogenous ion channels, ion channels and channel-linked receptors, or ion transporters may be used, it is preferable to use cells transformed or transfected with heterologous nucleic acids encoding such ion channels, ion channels and channel-linked receptors, or ion transporters so as to express predominantly a single type of ion channel, ion channel and channel-linked receptor, or ion transporter.

Preferred cells for heterologous cell surface protein expression are those that can be readily and efficiently transfected to express ion channels, ion channels and channel-linked receptors, or ion transporters. Cells that express native ion channels and cells which may be transfected to express ion channels, ion channels and channel-linked receptors, or ion transporters, are known to those of skill in the art, or may be identified by those of skill in the art. Many cells that may be genetically engineered to express a heterologous cell surface protein are known. Types of cells that can be used to express ion channel, ion channel and channel-linked receptors, or ion transporters include, but are not limited to, bacterial cells, yeast cells and mammalian cells. Examples of such cells include, but are not limited to, human embryonic kidney (HEK) cells, a HEK 293 cells (U.S. Pat. No. 5,024,939; Stillman et al., 1985, Mol. Cell Biol. 5, 2051-2060), Chinese hamster ovary (CHO) cells (ATCC Nos. CRL9618, CCL61, CRL9096), Xenopus laevis oocyte, (XLO) cell, baby hamster kidney (BHK) cells (ATCC No. CCL10), mouse L cells (ATCC No. CCLI.3), Jurkats (ATCC No. TIB 152) and 153 DG44 cells (Chasin (1986) Cell. Molec. Genet. 12: 555) human embryonic kidney (HEK) cells (ATCC No. CRL1573), PC12 cells (ATCC No. CRL17.21) and COS-7 cells (ATCC No. CRL1651).

The cells can be grown in solution or on a solid support. The cells can be adherent or non-adherent. Solid supports may include, but are not limited to, glass or plastic culture dishes, or multi-well plates.

Although any number of cells capable of eliciting a detectable fluorescence signal in an assay may be used in a multi-well plate, the number of cells seeded into each well may be chosen so that the cells are at or near confluence, but not overgrown, when the assays are conducted, so that the signal-to-background ratio of the signal is increased.

Alternatively, the methods of the invention can be performed using membranes (e.g., membrane vesicles) having ion channels, ion channels and channel-linked receptors, or ion transporters, rather than whole cells. The use of membrane vesicles are known to those of skill in the art.

The methods of the present invention can be applied to ion channels, channel-linked receptors, such as a receptor (e.g., GPCR), signal transduction pathways that are linked to or able to modulate the activity of an ion channel and proteins that are linked to ion channels, bacterial porins, or ion transporters.

Ion Channels

Types of ion channels that can be used in the methods of the invention include, but are not limited to, ligand- or voltage-gated, stretch-activated cation channels, selective or non-selective cation channels.

Types of ligand-gated non-selective cation channels include, but are not limited to, acetylcholine receptors, glutamate receptors such as AMPA, kainate, and NMDA receptors, 5-hydroxytryptamine-gated receptor-channels, ATP-gated (P2X) receptor-channels, nicotinic acetylcholine-gated receptor-channels, vanilloid receptors, ryanodine receptor-channels, IP.sub.3 receptor-channels, cation channels activated in situ by intracellular cAMP, and cation channels activated in situ by intracellular cGMP.

Types of voltage-gated ion channels include Ca²⁺, K⁺, and Na⁺. The channels can be expressed exogenously or endogenously. The channels can be stably or transiently expressed in both native or engineered cell lines.

Types of K⁺ channels include but, are not limited to, KCNQ1 (KvLOT1), KCNQ2, KCNQ3, KCNQ4, KCNQ5, HERG, KCNE1 (IeK, MinK), Kv1.5, Kir 3.1, Kir 3.2, Kir 3.3, Kir 3.4, Kir 6.2, SUR2A, ROMK1, Kv2.1, Kv1.4, Kv9.9, Kir6, SUR2B, KCNQ2, KCNQ3, GIRK1, GIRK2, GIRK3, GIRK4, h1K1, KCNA1, SUR1, Kv1.3, HERG (Conley, E. C. and Brammer, W. J.; 1999, The Ion Channel, Factsbook IV. Voltage-Gated Channels, Academic Press, London, UK), intracellular calcium-activated K⁺ channels, rat brain (BK2) (McKinnon, D. (1989) J. Biol Chem. 264, pp. 9230-8236); mouse brain (BK1) (Tempel, et al., (1988) Nature 332, pp. 837-839) and the like.

Types of Na⁺ channels include, but are not limited to, rat brain I and II (Noda, et al., 1986, Nature 320, pp. 188-192); rat brain III (Kayano, et al., 1988, FEBS Lett. 228, pp. 187-194); human II (ATCC No. 59742, 59743 and Genomics 1989, 5:204-208) and the like.

Types of Ca²⁺ channels include, but are not limited to, human calcium channel α₁, α₂, β and/or γ subunits (U.S. application Ser. Nos. 07/745,206 and 07/868,354), the ryanodine receptor (RyR) and the inositol 1,4,5-triphosphate receptor (IP_{3R}) (T. Jayaraman et al., J. Biol. Chem., 267, pp. 9474-77 (1992); A. M. Cameron et al., Proc. Natl. Acad. Sci. USA, 92, pp. 1784-44 (1995)), rabbit skeletal muscle α₁ subunit (Tanabe, et al., (1987) Nature 328, pp. 313-E318); rabbit skeletal muscle α₂ subunit (Ellis, et al., (1988) Science 241, pp. 1661-1664); rabbit skeletal muscle p subunit (Ruth, et al., (1989) Science 245, pp. 1115-1118); rabbit skeletal muscle gamma subunit (Jay, et al., (1990) Science 248, pp. 490-492) and the like.

Channel-Linked Receptors

The methods of the present invention can also be applied to indirectly measure the activity of channel-linked receptors and signal transduction systems. In an embodiment of the methods of the invention, the activity of channel-linked receptors is determined, where the activation of the receptor initiates subsequent intracellular events that lead to the modulation of ion channel activity. This modulation may result from interactions between receptor subunits with ion channels (e.g., GPCR β-γ subunits and GPCR-linked K⁺ channels (e.g., GIRKs)) or by changes in the concentrations of messenger molecules such as calcium, lipid metabolites, or cyclic nucleotides which, modulate the ion channel activity.

Among G-protein-coupled receptors muscarinic acetylcholine receptors (mAChR), adrenergic receptors, serotonin receptors, dopamine receptors, angiotensin receptors, adenosine receptors, bradykinin receptors, metabotropic excitatory amino acid receptors and the like, may be used.

Another type of indirect assay of the invention involves determining the activity of receptors which, when activated, result in a change in the level of intracellular cyclic nucleotides, e.g., cAMP, cGMP. For example, activation of some dopamine, serotonin, metabotropic glutamate receptors and muscarinic acetylcholine receptors results in an increase or decrease in the cAMP or cGMP levels of the cytoplasm. Furthermore, there are cyclic nucleotide-gated ion channels, e.g., rod photoreceptor cell channels and olfactory neuron channels (Altenhofen, W. et al., (1991) Proc. Natl. Acad. Sci U.S.A. 88:9868-9872 and Dhallan et al., (1990) Nature 347:184-187), that are permeable to cations upon activation by binding of cAMP or cGMP. Thus, in accordance with the methods of the present invention, a change in cytoplasmic ion levels, caused by a change in the amount of cyclic nucleotide activation of photo-receptor or olfactory neuron channels, is used to determine function of receptors that cause a change in cAMP or cGMP levels when activated. In cases where activation of the receptor results in a decrease in cyclic nucleotide levels, it may be preferable to expose the cells to reagents that increase intracellular cyclic nucleotide levels, e.g., forskolin, prior to adding a receptor-activating compound to the cells in the assay.

Cells used for this type of assay can be generated by co-transfection of a host cell with DNA encoding an ion channel (such as GIRK) and DNA encoding a channel-linked receptor (e.g., certain metabotropic glutamate receptors, muscarinic acetylcholine receptors, dopamine receptors, serotonin receptors and the like) which, when activated, cause a change in cyclic nucleotide levels in the cytoplasm.

Any cells expressing a receptor protein which is capable, upon activation of the receptor, of causing a change in the activity of an ion channel expressed in the cell may be used in the methods of the invention. For example, cells expressing a receptor protein which is capable, upon activation, of directly increasing the intracellular concentration of calcium (e.g., G-protein-coupled receptors), such as by opening gated calcium channels, or indirectly affecting the concentration of intracellular calcium by causing initiation of a reaction which utilizes Ca²⁺ as a second messenger, may be used in the methods of the invention. Cells endogenously expressing such channel-linked receptors or ion channels, and cells which may be transfected with a suitable vector encoding one or more such cell surface proteins, are known to those of skill in the art, or may be identified by those of skill in the art.

Receptors for use in the invention, include, but are not limited to, muscarinic receptors, e.g., human M2 (GenBank accession #M16404); rat M3 (GenBank accession #M16407); human M4 (GenBank accession 4M16405); human M5 (Bonner, et al., (1988) Neuron 1, pp. 403-410); and the like; neuronal nicotinic acetylcholine receptors, e.g., the human α₂, human α₃, and human β₂, subtypes disclosed in U.S. Ser. No. 504,455 (filed Apr. 3, 1990, which is hereby expressly incorporated by reference herein in its entirety); the human α₅, subtype (Chini et al., (1992) Proc. Natl. Acad. Sci. U.S.A. 89:1572-1576), the rat α₂ subunit (Wada, et al., (1988) Science 240, pp. 330-334); the rat α₃ subunit (Boulter, et al., (1986) Nature 319, pp. 368-374); the rat α₄ subunit (Goldman, et al., (1987) Cell 48, pp. 965-973); the rat α₅ subunit (Boulter, et al., (1990) J. Biol. Chem. 265, pp. 4472-4482); the chicken α₇ subunit (Couturier et al., (1990) Neuron 5:847-856); the rat β₂ subunit (Deneris, et al., (1988) Neuron 1, pp. 45-54) the rat β₃ subunit (Deneris, et al., (1989) J. Biol. Chem. 264, pp. 6268-6272); the rat β₄ subunit (Duvoisin, et al., (1989) Neuron 3, pp. 487-496); combinations of the rat α a subunits, rat NMDAR1 receptor (Moriyoshi et al., (1991) Nature 354:31-37 and Sugihara et al., (1992) Biochem. Biophys. Res. Comm. 185:826-832); mouse NMDA el receptor (Meguro et al., (1992) Nature 357:70-74); rat NMDAR2A, NMDAR2B and NMDAR2C receptors (Monyer et al., (1992) Science 256:1217-1221); rat metabotropic mGluR1 receptor (Houamed et al., (1991) Science 252:1318-1321); rat metabotropic mGluR2, mGluR3 and mGluR4 receptors (Tanabe et al., (1992) Neuron 8:169-179); rat metabotropic mGluR5 receptor (Abe et al., (1992) J. Biol. Chem. 267:13361-13368) and the like; adrenergic receptors, e.g., human beta 1 (Frielle, et al., (1987) Proc. Natl. Acad. Sci. 84, pp. 7920-7924); human alpha 2 (Kobilka, et al., (1987) Science 238, pp. 650-656); hamster beta 2 (Dixon, et al., (1986) Nature 321, pp. 75-79) and the like; dopamine receptors, e.g., human D2 (Stormann, et al., (1990) Molec. Pharm. 37, pp. 1-6); mammalian dopamine D2 receptor (U.S. Pat. No. 5,128,254); rat (Bunzow, et al., (1988) Nature 336, pp. 783-787) and the like; serotonin receptors, e.g., human 5HT1a (Kobilka, et al., (1987) Nature 329, pp. 75-79); serotonin 5HT1C receptor (U.S. Pat. No. 4,985,352); human 5HT.sub.1D (U.S. Pat. No. 5,155,218); rat 5HT2 (Julius, et al., (1990) PNAS 87, pp.928-932); rat 5HT1c (Julius, et al., (1988) Science 241, pp. 558-564) and the like.

Ion transporters

The methods of the present invention can also be applied to measure the activity of ion transporters.

Ion transporters for use in the invention, include, but are not limited to, neurotransmitter ion transporters (e.g., dopamine ion transporter, glutamate ion transporter or serotonin ion transporter) (Gadea, A. and Lopez-Colome, A. M., J. Neurosci. Res., 2001, 63, 453-460) sodium-potassium ATPase, proton-potassium ATPase (Silver, R. B. and Soleimani, M., Am. J. Physiol., 1999, 276, F799-F811), sodium/calcium exchanger, and potassium-chloride ion co-transporter (Gillen, C. M. et al., J. Biol. Chem., 1996, 271, 16237-16244).

Buffers

Types of buffer for use in the methods of the invention can be any buffer with buffering capacity of about pH 5.5 to 9.0, such as HEPES and PBS. Buffers are well known in the art and can be readily obtained in Molecular Cloning; A Laboratory Manual (2nd edition, Sambrook, Fritch, and Maniatis 1989, Cold Spring Harbor Press) or in Short Protocols in Molecular Biology (Ausubel, F. M., et al., 1989, John Wiley & Sons).

Although it is possible to perform all the assays in known physiological Cl⁻ containing buffers, the novel Cl⁻-free buffer conditions and low Cl⁻ cell growth medium produce more robust and consistent results.

Novel Cell Growth Medium

In an embodiment of the invention, a novel cell growth medium and assay buffer solution are provided, to permit the use of higher concentrations of thallium ions in solution for more consistent assay results. In both these solutions, a thallium ions concentration of up to 200 mM, can be used.

The novel cell growth medium also includes very low levels of Cl⁻ down to nearly complete absence of Cl⁻. The cell growth medium includes all the components (cations, anions, vitamins, and amino acids), suitable for growing cells, as known in the art, except that the Cl⁻ concentration has been limited to no more than approximately 2 mM. The remainder of the Cl⁻ can be replaced with the organic anion gluconate. Any buffer with buffering capacity of about pH 5.5 to 9.0, such as HEPES, can be used.

The cell growth media may include of one or more of the following: sodium gluconate; potassium gluconate; MgSO₄O.7H₂O; NaHCO₃; calcium gluconate; NaH₂PO₄; glucose; vitamins; amino acids; glutamine and buffer (for example, HEPES).

A preferred embodiment of the novel cell growth media composition includes sodium gluconate (109 mM); potassium gluconate (5.4 mM); MgSO₄7H₂O (0.8 mM); NaHCO₃ (26.2 mM); calcium gluconate (3.6 mM); NaH₂PO₄ (1.2 mM); HEPES, pH 7.3 w/NaOH (25 mM); Glucose (5.6 mM); 100X Vitamins (10 ml/l); 50X amino acids (20 ml/l); and glutamine (2 mM).

Novel Cl⁻-free Assay Buffer

The present invention also provides for compositions and methods of use of novel Cl³¹ -free assay buffers. The Cl⁻-free assay buffer is any buffer in which the Cl⁻ ion concentration has been limited to approximately 2 mM. The remainder of the Cl⁻ ion can be replaced with the organic anion gluconate. The novel Cl⁻-free assay buffer composition may include a range of osmolality from 250 to 360 mOsM and a buffering capacity from pH 5.5 to pH 9.0. The osmolality of the Cl⁻-free assay buffer is dependent upon the cell type used in the methods of the invention. For example, cells such as Xenopus oocytes can survive under conditions of below 200 mOsM, while other cell types may survive under conditions of high osmolalities, of up to 1000 mOsM of cell growth media, and assay buffers.

The novel Cl⁻-free assay buffer may include sodium gluconate, potassium gluconate, calcium gluconate, magnesium gluconate, glucose, and buffer (for example, HEPES). A preferred embodiment of the novel Cl⁻-free assay buffer composition includes sodium gluconate (140 mM), potassium gluconate (2.5 mM), calcium gluconate (6 mM), magnesium gluconate (1 mM), glucose (5.6 mM) and HEPES (10 mM).

Thallium Salts

In the methods of the invention, thallium ion (i.e., tracer) flux across the cell membrane is measured using thallium sensitive agents. Solutions of thallium salts provide the thallium ions.

The thallium salts for use in thallium solutions used in the methods of the invention include those that are water soluble, such as, Tl₂SO₄, Tl₂CO₃, TlCl, TlOH, TlOAc, TlNO₃ salts and the like.

Thallium Sensitive Agents

The methods of the invention provide signal generating thallium sensitive agents.

Thallium sensitive agents are employed as an indicator of the flux of thallium across the cell membrane and are sufficiently sensitive so as to produce detectable changes in fluorescence or optical intensity in response to changes in the concentration of the thallium ions in the cell cytoplasm. Types of thallium sensitive agents that can produce a detectable signal include, but are not limited to, fluorescent compounds and non-fluorescent compounds.

Thallium Sensitive Fluorescent Agents

An embodiment of the invention for the thallium sensitive agent is a fluorescent compound. Essentially any thallium-sensitive fluorescent compound that can be loaded into cells can be used. Preferably, the compound is selected to detect low concentrations of thallium ions. These fluorescent compounds can either show a decrease or an increase in fluorescence in the presence of thallium ions.

Suitable types of thallium sensitive fluorescent agents include, but are not limited to ANTS, Fluo-4, Fluo-3, PBFI, Phen Green, Magnesium Green, BTC, APTRA-BTC, Mag-Fura Red Fluo-4FF, FluoZin-1 and FluoZin-2 are suitable dyes (Molecular Probes Inc., Eugene, Oreg.). ANTS, Fluo-4, Fluo-3, PBFI, Phen Green, APTRA-BTC and Mag-Fura Red show decrease fluorescence in the presence of thallium ions. Magnesium Green, BTC, Fluo-4FF, FluoZin-1 and FluoZin-2 show fluorescence that is increased by thallium ions. The thallium sensitive fluorescent agents may be hydrophilic or hydrophobic.

The thallium sensitive fluorescent agents are loaded into the cytoplasm by contacting the cells with a solution comprising a membrane-permeable derivative of the dye, however, the loading process may be facilitated, where a more hydrophobic form of the indicator is used. Thus, fluorescent indicators are known and available as more hydrophobic acetoxymethyl esters (AM) which are able to permeate cell membranes much more readily than the unmodified dyes. As the acetoxymethyl ester form of the dye enters the cell, the ester group is removed by cytosolic esterases, thereby trapping the dye in the cytosol.

The fluorescence of the thallium sensitive agent is measured by devices that detect fluorescent signals. One type of device is a FLIPR (Molecular Devices Corp., Sunnyvale, Calif.), where fluorescence is recorded at a rate of up to 1 Hz, before, during, and after addition of thallium ions, and addition of candidate ion channel, channel-linked receptor or ion transporter modulators. Example of devices used for non-adherent cells include the FLIPR and flow cytometer (Becton-Dickenson).

In an embodiment of the invention for detecting modulators of ion channel activity, BTC is the thallium sensitive fluorescent agent. In the presence of thallium ion, BTC shows a strong increase in fluorescence, when excited at 488 nm.

The transport of thallium sensitive agents and thallium ions into cells is followed by an increase or decrease in the signal. Thallium ions moves through open channels along their concentration gradient and change the intensity of dye fluorescence inside the cell, resulting in the recorded signals. Activation of the cation channel enhances the rate of influx of thallium ions (resulting in a change in the fluorescence of the thallium sensitive fluorescent compound) and inhibition decreases the rate of influx of thallium ions (resulting in no or little change in the fluorescence of the thallium sensitive fluorescent agent). Generally the fluorescence remains the same if no thallium ion is bound to it. Thus if the ion channel is blocked by the candidate channel modulator and thallium influx is inhibited, little or no change in fluorescence is detected.

Extracellular Ouenchers

In an embodiment where a fluorescent thallium sensitive agent is used, the excess fluorescent compound is removed by using a sufficient amount of an extracellular quencher. The use of extracellular quencher obviates the need to wash unloaded thallium sensitive fluorescent agent from the cells. The extracellular quenchers are not cell permeant and can be light absorbing fluorescent compounds having a fluorescence which can be easily separated from that of the thallium sensitive fluorescent agent. The absorption spectrum of the extracellular quenchers significantly absorbs the emission of the thallium sensitive fluorescent agent. The extracellular quenchers must be of a chemical composition that prevents their passage into the cells, and generally the quenchers should be charged or very large compounds. The concentration range for extracellular quenchers will range from micromolar to millimolar, depending on their light absorbing properties. Types of extracellular quenchers that can be used include, but are not limited to, tartrazine and amaranth, or a mixture of such quenchers. Quenchers are described in the Sigma-Aldrich Handbook of Dyes, Stains, and Indicators (Floyd G. Green, 1990, St. Louis, Mo.).

Thallium Sensitive Non-Fluorescent Agents

The method of the invention further provides thallium sensitive non-fluorescent agents. One embodiment of the invention provides using a thallium sensitive agent which is a non-fluorescent compound that reacts with thallium ion to form a product that can either form a precipitate or form a product that is colored, and thus cause detectable changes in the optical density of the test mixture. These compounds include but are not limited to iodide, bromide, and chromate.

In an embodiment of the invention, in which the thallium sensitive agent is a non-fluorescent compound, absorbance can be recorded by a spectrophotometer, before, during, and after addition of thallium ions, and addition of channel modulators. The cells expressing ion channels and/or receptors are loaded with iodide, bromide or chromate ion. The cells are washed with, for example, a buffered saline solution. The transport of thallium into cells causes an increase or decrease in the optical density signal. Thallium ions pass through open channels down its concentration gradient and changes the optical density inside the cell, resulting in the recorded signals. Activation of the cation channel enhances the rate of influx of thallium ions (resulting in an increased formation of precipitant or colored product) and inhibition decreases the rate of influx of thallium ions (resulting in no or little change in precipitation or colored product formation). Generally the optical density remains the same if no thallium ions reacts with the non-fluorescent compound. Thus if the ion channel is blocked, and thallium ions influx is inhibited, little or no change in optical density is detected.

Candidate Modulators

The invention provides methods for identifying compounds that modulate ion channel, channel-linked receptor, or ion transporter activity. Essentially any chemical compound can be used as a potential modulator in the assays of the invention, although compounds that can be dissolved in aqueous or organic (especially DMSO-based) solutions, are preferred. It will be appreciated by those of skill in the art that there are many commercial suppliers of chemical compounds, including Sigma Chemical Co. (St. Louis, Mo.), Aldrich Chemical Co. (St. Louis, Mo.), Sigma-Aldrich (St. Louis, Mo.), Fluka Chemika-Biochemica Analytika (Buchs, Switzerland), and the like.

Examples of ion channels, channel-linked receptors and ion transporters have been provided above.

High-Throughput Screening Methods

The methods of the invention can be adapted for high-throughput screening. High-throughput screening assays are known, and employ the used of microtiter plates or pico-nano- or micro-liter arrays.

The high-throughput methods of the invention are performed using whole cells expressing ion channels, ion channel and channel-linked receptors or ion transporters of interest, using the following steps of 1) growing the cells under suitable conditions; 2) optionally, adhering the cells onto solid support; 3) loading the cells with a cell permeant thallium sensitive agent that produces a detectable signal; 4) treating the cells under suitable conditions (washing or adding extracellular quenchers) to remove excess thallium sensitive agent; 5) measuring the detectable signal; 6) adding a solution containing thallium ions and appropriate stimulus solution; 7) adding a candidate modulatory compound; 8) measuring detectable signal; and 9) recording the changes in the detectable signal (i.e., before and after the addition of thallium ions, stimulus solution and modulatory compound). The change in the detectable signal indicates the effect of the channel modulators.

The assays of the invention are designed to permit high throughput screening of large chemical libraries, e.g., by automating the assay steps and providing candidate modulatory compounds from any convenient source to assay. Assays which are run in parallel on a solid support (e.g., microtiter formats on microtiter plates in robotic assays) are well known. Automated systems and methods for detecting and measuring changes in optical detection (or signal) are known (U.S. Pat. No. 6,171,780; 5,985,214; 6,057,114).

The high throughput screening methods of the invention include providing a combinatorial library containing a large number of potential therapeutic modulating compounds (Borman, S, C. & E. News, 1999, 70(10), 33-48). Preparation and screening of combinatorial chemical libraries is well known to those of skill in the art.

A combinatorial chemical library is a collection of diverse chemical compounds generated by using either chemical synthesis or biological synthesis, to combine a number of chemical building blocks, such as reagents. For example, a linear combinatorial chemical library, such as a polypeptide library, is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Such combinatorial chemical libraries include, but are not limited to, peptide libraries (see, e.g., U.S. Pat. No. 5,010,175, Furka, Int. J Pept. Prot. Res., 1991, 37:487-493 and Houghton, et al., Nature, 1991, 354, 84-88). Other chemistries for generating chemical diversity libraries can also be used. Such chemistries include, but are not limited to, peptides (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 1993, 90, 6909-6913); vinylogous polypeptides (Hagihara, et al., J. Amer. Chem. Soc. 1992, 114, 6568); nonpeptidal peptidomimetics with beta-D-glucose scaffolding (Hirschmann, et al., J. Amer. Chem. Soc., 1992, 114, 9217-9218); analogous organic syntheses of small compound libraries (Chen, et al., J. Amer. Chem. Soc., 1994, 116, 2661; Armstrong, et al., Acc. Chem. Res., 1996, 29, 123-131); or small organic molecule libraries (see, e.g., benzodiazepines, Baum C&E News, Jan. 18,1993, page 33,); oligocarbamates (Cho, et al., Science, 1993, 261, 1303); and/or peptidyl phosphonates (Campbell, et al., J. Org. Chem. 1994, 59, 658); nucleic acid libraries (see, Seliger, H et al., Nucleosides & Nucleotides, 1997, 16, 703-710); peptide nucleic acid libraries (see, e.g., U.S. Pat. No. 5,539,083); antibody libraries (see, e.g., Vaughn, et al., Nature Biotechnology, 1996, 14(3), 309-314 and PCT/US96/10287); carbohydrate libraries (see, e.g., Liang, et al., Science, 1996, 274, 1520-1522 and U.S. Pat. No. 5,593,853, Nilsson, U J, et al., Combinatorial Chemistry & High Throughput Screening, 1999 2, 335-352; Schweizer, F; Hindsgaul, O. Current Opinion In Chemical Biology, 1999 3, 291-298); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514) and other similar art.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

The combinatorial chemical libraries are screened in one or more assays, as described herein, to identify library members (particular chemical species or subclasses) that display the ability to modulate the target ion channel activity (Borman, S., supra; Dagani, R. C. & E. News, 1999, 70(10), 51-60), channel-linked receptor or ion transporter activity. The modulating compounds thus identified can serve as conventional lead compounds or can themselves be used as potential or actual therapeutics.

In high throughput assays it is desirable to run positive controls to ensure that the components of the assays are working properly. In an example of a positive control, a known cation channel opener compound is contacted with the sample mixture of the assay, and the resulting increase in cation channel activity is determined according to the methods herein. In another example of a positive control, for cells expressing cation channels, a known cation channel blocker compound can be added, and the resulting decrease in cation channel activity is similarly detected. It will be appreciated that candidate modulators can also be combined with compounds having known effects on ion channels, channel-linked receptors, or ion transporters. For example, known cation channel openers or blockers can be used to find modulators, which further effect the cation channel activation or suppression, that is otherwise caused by the presence of the known ion channel modulator.

In the high throughput assays of the invention, it is possible to screen up to several thousand different candidate modulators in a single day. In particular, each well of a microtiter plate can be used to run a separate assay against a selected potential modulator, or if concentration or incubation time effects are to be observed, every 5-10 wells can test a single modulator. Thus, a single standard microtiter plate can assay about 100 (96) modulators. If 1536 well plates are used, then a single plate can easily assay from about 100 to about 1500 different compounds. It is possible to assay many different plates per day. Assay screens for up to about 6,000-20,000, and even up to about 100,000-1,000,000 different candidate modulator compounds are possible using the methods of the invention.

Advantages of the Invention

The invention represents an improvement over present technology, for detecting and characterizing modulators of ion channels, channel-linked receptors or ion transporters, in various ways. For example, (a) there are no requirements for radioactive reagents; (b) the methods take advantage of the permeability of thallium ions; (c) the activity of the ion channel, channel-linked receptors or ion transporters is monitored solely by the thallium ion flux and is not perturbed by the presence of physiologically relevant ions; (d) there is no requirement for chemical or biochemical modification of the ion channels, channel-linked receptors or ion transporters; (e) the assays can be performed in whole cells, specifically with the use of the novel low Cl⁻ cell growth medium and novel Cl⁻-free assay buffer; (f) the signal or emission generated by the assay is significantly larger and more robust than that typically obtained using previously known optical methodologies; (g) a change in signal is generated by the presence of a candidate modulator, thus facilitating the identification of specific modulatory agents; (h) there are a large variety of thallium sensitive agents that are currently available; (i) the assay format does not require that the ion channel and/or receptor be immobilized on a solid support during the course of the assay; and (j) each of the formats described is readily amenable for automation and high-throughput screening.

The following Examples demonstrate the methods and compounds of the present invention and assist one of ordinary skill in making and using the same. The Examples are not intended in any way to otherwise limit the scope of the disclosure of the protection granted by Letters Patent granted hereon.

### EXAMPLE I

Example I describes expression of ion channels of interest in mammalian cells.

TABLE I

| Clone | | | Cell Line/ |
|---|---|---|---|
| ID/Construct | Restriction Sites | Vector | Antibiotic Conc. |
| hSlo/BK | 5′HindIII/3′BamHI | pCDNA3 | HEK293/G418 800 |
| | | | μg/ml |
| mKCNQ2 | 5′Blunt-ended/ | pCDNA3 | HEK293/G418 800 |
| | 3′NotI | | μg/ml |
| hSK2 | 5′EcoRi/3′EcoRI | pCDNA3 | HEK293/G418 800 |
| | | | μg/ml |
| hVR1 | 5′ and 3′ | pIRESneo | CHO/G418 500 |
| | Blunt-ended into EcoRV | | μg/ml |
| | site | | |

Table I displays the DNA constructs used in the thallium sensitive assays in the examples. Restriction sites for each of the cloned illustrate how the ion channel cDNAs of interest were subcloned into the DNA vector (pCDNA3 (Invitrogen, Carlsbad, Calif.) and pIRESneo (Clonetech, Palo Alto, Calif.)) required for mammalian cell expression. The cell types (HEK; human embryonic kidney cells) and concentration of antibiotic used in the selection and preparation of stable cell lines are indicated. Standard molecular biology methodologies were utilized in the cloning of the ion channel genes listed in Table I. Detailed cloning strategies are also described in the art (HSlo/BK (Dworetzky, SI., et al., Mol. Brain Res. 27: 189-193); KCNQ2 (Patent WO 99/07832); SK channels (Kohler M, et al., Science 1996, 273:1709-1714); and VR1 (M J Caterina, et al., Nature 1997, 389:816-824)).

VR1-pIRESneo was transfected into CHO cells using Lipofectamine PLUS (Life Technologies) transfection kit protocol. hSK-pCDNA3, hslo(BK)-pCDNA3, and mKCNQ2-pCDNA3 were transfected separately into HEK-293 cells using Lipofectamine PLUS (Life Technologies) transfection kit protocol. Cells were selected using G418 (Life Technologies) at a concentration of 500 82 g/ml for CHO cells and 800 μg/ml for HEK-293 cells. After 12 days of drug selection each cell line was analyzed for channel expression using the thallium influx assay, as described herein (see Example II). The hVR1 expressing CHO cells were also evaluated for the channel's ability to increase intracellular calcium using the calcium-sensitive dye fluo-3 according to the directions for measuring calcium responses in CHO cells, as described in the FLIPR manual (Molecular Devices, Sunnyvale, Calif.).

### EXAMPLE II

Example II demonstrates the ability of the thallium influx assay to measure the effect of a peptide inhibitor, Apamin (Sigma Chemical Co., St. Louis, Mo.; from bee venom), on small conductance calcium-activated K⁺ channels (SK2), (Kohler M, et al., Science. 1996, 273:1709-14), using changes in BTC fluorescence, as a measure of thallium influx.

A HEK-293 cell line (obtained from ATCC, Manassas, Va.) stably expressing the small conductance calcium-activated K⁺ channel (SK2) was seeded at .about.80% confluence in a 384 well microtiter plate, coated with poly-D-lysine plates, containing 20 μl/well low Cl⁻ cell growth medium. The cells were allowed to incubate overnight at 37 C in a 5% CO₂ incubator.

The cell-containing plates were removed from the incubator and loaded for approximately 15 min with 2 μM BTC-AM (Molecular Probes, Eugene, Oreg.) dissolved in 20 μL/well Cl⁻-free assay buffer containing amaranth and tartrazine (the final concentration in the assay is 2 mM amaranth and 1 mM tartrazine). The AM ester of BTC (BTC-AM) is membrane permeant. As it diffuses across the membrane, it is cleaved by cellular esterases, producing a charged, membrane impermeant dye, BTC. These types of dyes and loading mechanisms are well known to those familiar with the art.

Once loaded, apamin (5 μl/well of 500 nM stock dissolved in Cl⁻-free assay buffer (Table II) or an equivalent volume of Cl⁻-free assay buffer) was added. The microtiter plates were then transferred to the plate reader, FLIPR. After, or coincident with exposure to apamin or Cl⁻-free assay buffer alone, the cells were exposed to 5 μl/well of a stimulus buffer containing 5 μM ionomycin (Calbiochem) and 7.5 mM Tl₂SO₄ dissolved in Cl⁻-free assay buffer containing 2 mM amaranth and 1 mM tartrazine.

All data shown were collected using the FLIPR (Molecular Devices, Sunnyvale, Calif). The preferred standard protocol acquires data at 1 Hz for 1 min. 10 seconds of baseline prior to addition of the stimulus buffer.

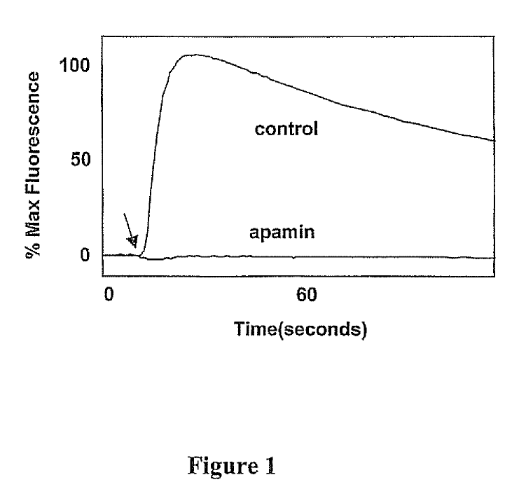

The ability of the thallium influx technique to allow the measurement of the activation and blockade of SK2, small-conductance, calcium-activated K⁺ channels was demonstrated as shown in FIG. 1.

A stable baseline fluorescence was observed for cells incubated in the presence or absence of the SK2 channel blocker apamin, prior to the addition, of the calcium ionophore, ionomycin, and thallium ions. Upon the addition of thallium ions plus ionomycin (which causes an increase in intracellular calcium and subsequent activation of SK2) a substantial increase in BTC fluorescence was observed. This increase in fluorescence was completely abolished by the addition of the SK2 blocker, apamin. Since an apamin-sensitive increase in BTC fluorescence was not observed in native, untransfected HEK cells under identical conditions, or with the addition of thallium in the absence of ionomycin, these results clearly demonstrate the ability of the thallium influx assay to measure the activation of SK2 channels. These data also demonstrate the ability of the assay to identify blockers of calcium-activated SK2 channels.

All the reagents used in the cell growth media and assay buffer were obtained from Sigma-Aldrich (St. Louis, Mo.), except for the 100X vitamin and 50X amino acid solution which were obtained from Life Technologies (Rockville, Md.). The designations used for the reagents were provided from the supplier.

TABLE II
Low CI⁻growth medium (290-300 mOsM)

| Component | Concentration (mM) | Concentration (g/l) |
|---|---|---|
| Na gluconate | 109 | 23.77 |
| K gluconate | 5.4 | 1.26 |
| MgSO₄•7H₂O | 0.8 | 0.2 |
| NaHCO₃ | 26.2 | 2.2 |
| Calcium gluconate | 3.6 | 0.78 |
| NaH₂PO4 | 1.2 | 0.17 |
| HEPES, pH 7.3 w/NaOH | 25 | 5.96 |
| Glucose | 5.6 | 1.0 |
| 100× Vitamins | 10 (ml/l) | |
| 50× amino acids | 20 (ml/l) | |
| glutamine | 2 | 0.292 |

TABLE III
CI⁻free Assay Buffer (290-300 mOsM)

| Component | Concentration (mM) | Concentration (g/l) |
|---|---|---|
| Na gluconate | 140 | 30.53 |
| K gluconate | 2.5 | 0.59 |
| Calcium gluconate | 6.0 | 1.29 |
| Magnesium gluconate | 1.0 | 0.21 |
| HEPES, pH 7.3 w/NaOH | 10 | 2.38 |
| Glucose | 5 | 1.0 |

### EXAMPLE III

Example III demonstrates the use of the thallium influx assay of the invention to detect compounds that block or open Ca²⁺ sensitive, voltage-dependent Maxi-K channels using changes in BTC fluorescence as a measure of thallium influx.

All experimental conditions for this example were the same as Example II, with the following exceptions:
1. HEK-293 cells were stably transfected with Maxi-K channels. Cells expressing the large conductance calcium-activated K⁺ channel, Maxi-K (Dworetzky S I, Trojnacki J T, Gribkoff V K. Brain Res Mol Brain Res. 1994, 1:189-93) (a.k.a. BK, slo) were used; and
2. The channel opener used was NS-1619 (Sigma-Aldrich, St Louis, Mo.) at a final concentration of 15 μM. The channel blocker used was Iberiotoxin (Sigma-Aldrich, St. Louis, Mo.) at a final concentration of 100 nM.

To detect channel blockers, the assay was started by adding 11 μl of stimulus buffer containing: 15 μM ionomycin, 12.5 Tl₂SO₄ and 50 mM K₂SO₄ dissolved in the Cl⁻-free assay buffer (Table II) containing 2 mM amaranth and 1 mM tartrazine.

To detect channel openers, the stimulus buffer was identical to the assay conditions of the channel blockers with the exception that 5 μM ionomcyin was used in place of 15 μM ionomycin. Under these conditions the channels were submaximally opened, allowing observation of openers of the channels.

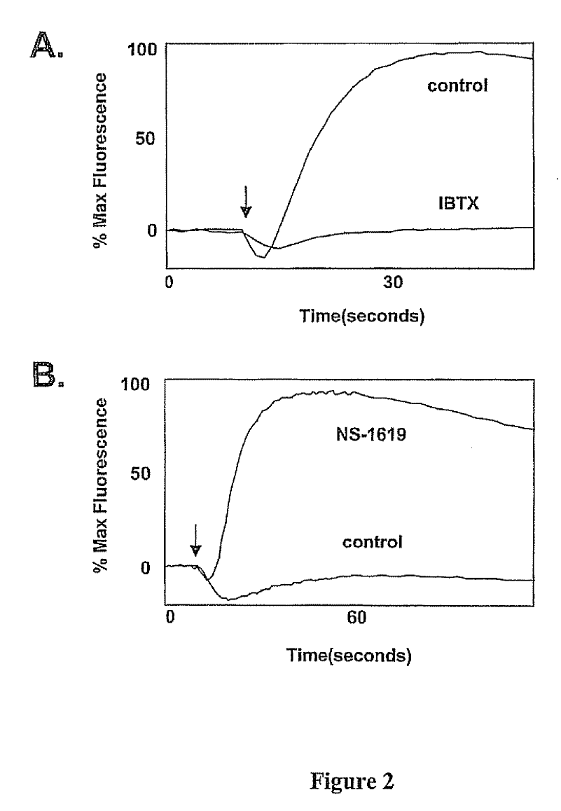

The ability of the thallium influx technique to allow the measurement of the activation and blockade of Maxi-K, large-conductance, calcium and voltage-dependent K⁺ channels was demonstrated as shown in FIG. 2.

A stable baseline fluorescence was observed for cells incubated in the presence or absence of the Maxi-K channel blocker iberiotoxin or the Maxi-K channel opener NS-1619, prior to the addition of ionomycin and supraphysiological potassium, to cause HEK cell depolarization, and thallium influx. Upon the addition of thallium, ionomycin, and potassium ions a substantial increase in BTC fluorescence was observed. This increase in fluorescence was completely abolished by the addition of iberiotoxin (FIG. 2A). Under slightly different conditions, which favored modest opening of the Maxi-K channel, the addition of NS-1619 caused a marked increase in the thallium-induced increase in BTC fluorescence compared to that observed in the absence of NS-1619 (FIG. 2B). Neither iberiotoxin nor NS-1619 had any effects on the fluorescence of BTC loaded into native, untransfected HEK cells. These results clearly demonstrate the ability of the thallium influx technique to identify both blockers and openers of calcium and voltage-dependant Maxi-K channels, and to measure the activity of these modulators.

### EXAMPLE IV

Example IV demonstrates the ability of the thallium influx assay to detect compounds that block or open the voltage-gated K⁺ channel KCNQ2 (European Patent No. WO 99/07832) using changes in BTC fluorescence as a measure of thallium influx.

All experimental conditions for this example were the same as Example II with the following exceptions:
1. HEK-293 cell line stably transfected with the voltage-gated K⁺ channel KCNQ2 was used;
2. The channel opener used was retigabine (Main, M. J., et al., Mol. Pharmacol., 2000, 58, 253-62) at a final concentration of 15 μM; and
3. The channel blocker DMP-543 (Zaczek, R., et al., J. Pharmacol. Exp. Ther. 1998, 285, 724-30) used was at a final concentration of 15 μM.

To detect channel blockers the KCNQ2 channels were opened with a combination of thallium ions (5 mM) and K⁺ (20 mM). To detect openers the assay was initiated with thallium ions (3 mM).

To detect channel blockers the assay was started by adding 11 μl of stimulus buffer containing: 12.5 mM Tl₂SO₄ and 50 mM K₂SO₄ dissolved in the Cl⁻-free assay buffer (Table II) containing 2 mM amaranth and 1 mM tartrazine.

To detect openers the assay was started by adding 11 μl of stimulus buffer containing: 7.5 mM Tl₂SO₄ dissolved in the Cl⁻-free assay buffer containing 2 mM amaranth and 1 mM tartrazine.

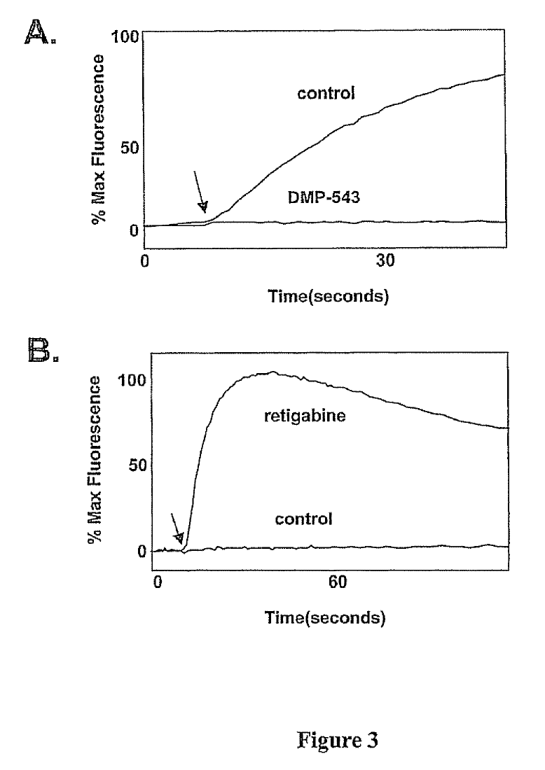

The ability of the thallium influx technique to allow the measurement of the activation and blockade of KCNQ2 voltage-gated K⁺ channels is shown in FIG. 3. A stable baseline fluorescence was observed for cells incubated in the presence or absence of the KCNQ2 channel blocker DMP-543 or the KCNQ2 channel opener retigabine, prior to the addition of thallium and supraphysiological potassium (to cause HEK cell depolarization). Upon the addition of thallium and potassium a substantial increase in BTC fluorescence was observed. This increase in fluorescence was completely abolished by the addition of DMP-543 (FIG. 3A). Under slightly different conditions which favor modest opening of the KCNQ2 channel the addition of the KCNQ2 opener, retigabine, caused a marked increase in the thallium-induced increase in BTC fluorescence compared to that observed in the absence of retigabine (FIG. 3B). Neither DMP-543 nor retigabine had any effects on the fluorescence of BTC loaded into native, untransfected HEK cells. These data clearly demonstrate the ability of the thallium influx technique to identify both blockers and openers of voltage-gated KCNQ2 channels and to measure the activity of these modulators.

### EXAMPLE V

Example V demonstrates the ability of thallium influx technique to detect modulators of the ligand-gated, non-selective cation channel, VR1 (capsaicin receptor) (Caterina M J, et al., Nature 1997, 389, 816-24) using changes in BTC fluorescence as a measure of thallium influx.

All experimental conditions for this example were the same as Example II with the following exceptions:
1. The cell line CHO stably expressing the non-selective cation channel vanilloid receptor (VR1) was used.
2. The channel antagonist, capsazepine (Sigma-Aldrich, RBI St Louis, Mo.) was applied at a final concentration of 10 μM; and
3. The assay was started by adding 5 μl of stimulus buffer containing: 1 μM capsaicin and 7.5 mM Tl₂SO₄ dissolved in a Cl⁻-free assay buffer containing 2 mM amaranth and 1 mM tartrazine.

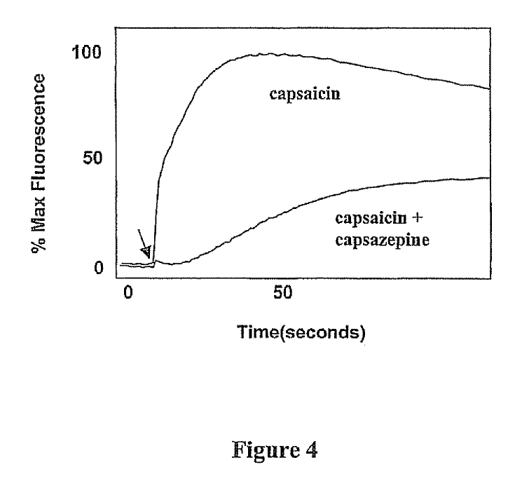

The ability of the thallium influx assay to allow the measurement of the activation and inhibition of VR1 ligand-gated, non-selective cation channels was demonstrated as shown in FIG. 4.

A stable baseline fluorescence was observed for cells incubated in the presence or absence of the VR1 antagonist, capsazepine, prior to the addition of thallium and the VR1 agonist, capsaicin. Upon the addition of thallium and capsaicin a substantial increase in BTC fluorescence was observed. This increase in fluorescence was completely abolished by the addition of capsazepine. Capsaicin alone causes a small decrease, but no increase, in BTC fluorescence VR1 expressing CHO cells due to BTC's calcium sensitivity. Capsazepine alone has no effect on the fluorescence of BTC loaded into VR1 expressing CHO cells. These data clearly demonstrate the ability of the thallium influx technique to identify both agonists and antagonists of the ligand-gated, non-selective cation channel VR1, and to measure the activity of these modulators.

### EXAMPLE VI

Example VI demonstrates the ability of the thallium efflux technique to detect inhibitors of the small conductance calcium-activated K⁺ channel (SK2).

All experimental conditions for this example were the same as Example II, with the following exception:
1. Instead of loading the cells with 2 μM BTC-AM, the cells were loaded with 2 μM FluoZin-1 (Molecular Probes, Eugene, Oreg.)

After loading the cells with FluoZin-1, the cells were exposed to 10 μl/well of Cl⁻-free assay buffer containing 7.5 mM Tl₂SO₄ for 10 minutes at room temperature. This step loads the cells with thallium which interacts with the thallium ion sensitive fluorescent dye FluoZin-1 and increases its fluorescence.

Following loading the cells with thallium ions, the solution bathing the cells was aspirated off, and replaced with 80 μl/well of Cl⁻-free assay buffer. The 80 μl/well of Cl⁻-free assay buffer was immediately aspirated off and replace with 40 μl/well of Cl⁺-free assay buffer containing amaranth and tartrazine at 2 mM and 1 mM, respectively.

Apamin (10 μl/well of 5 μM stock solution dissolved in Cl⁻-free assay buffer) or an equivalent volume of Cl⁻-free assay buffer without apamin was added, where appropriate, before transferring the thallium ions and FluoZin-1 loaded cells to the FLIPR for measurement.

To detect the activity of SK2 and the SK2 blocker apamin, the assay was started by the addition of 13 μl/well of stimulus buffer containing: 5 μM ionomycin dissolved in Cl⁻-free assay buffer. As a control, some wells were treated with Cl⁻-free assay buffer alone, without the addition of ionomycin.

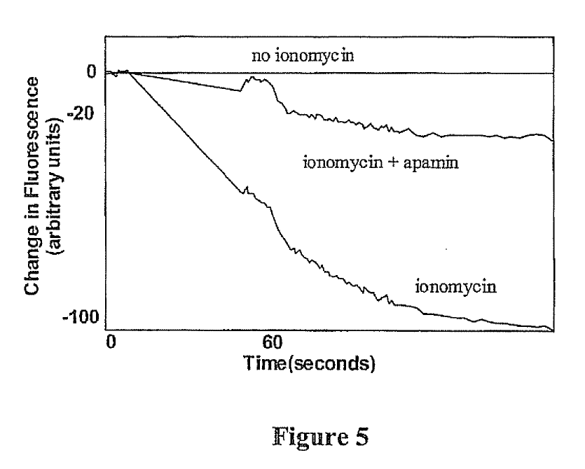

The ability to detect the activity of SK2 and its inhibition by apamin using the thallium efflux technique is shown in FIG. 5. A similar baseline fluorescence was observed in cells in the presence or absence of the SK2 blocker apamin. Upon the addition of ionomycin, a decrease in fluorescence was observed due to thallium ions dissociating from FluoZin-1 and exiting the cells via the activated SK channel. This decrease in fluorescence was absent without the addition of ionomycin and nearly abolished by the presence of the SK2 blocker apamin. These data clearly demonstrate the ability of the thallium efflux technique to detect the activity of SK2 and its inhibition by apamin.

### EXAMPLE VII

Example VII demonstrates the ability of the thallium influx technique to detect agonists and antagonist of the G-protein coupled receptor, Muscarinic acetylcholine receptor, through its activation of the small conductance calcium-activated K⁺ channel, SK2.

All experimental conditions for this example were the same as Example II, with the following exceptions:
1. The same SK2 expressing HEK-293 cells were used. HEK-293 cells natively express a muscarinic acetylcholine receptor.
2. Instead of pre-incubating selected wells with the SK2 blocker apamin, some wells were preincubated with 10 μM atropine, an acetylcholine receptor antagonist.

To detect the activity of an agonist of the muscarinic receptor, the assay was started by the addition of 13 μl/well of thallium containing stimulus buffer with: 10 μM of the muscarinic receptor agonist, oxotremorine-M (oxo-M), dissolved in Cl⁻-free assay buffer. As a control, some wells were treated with Cl⁻-free assay buffer alone without the addition of oxo-M.

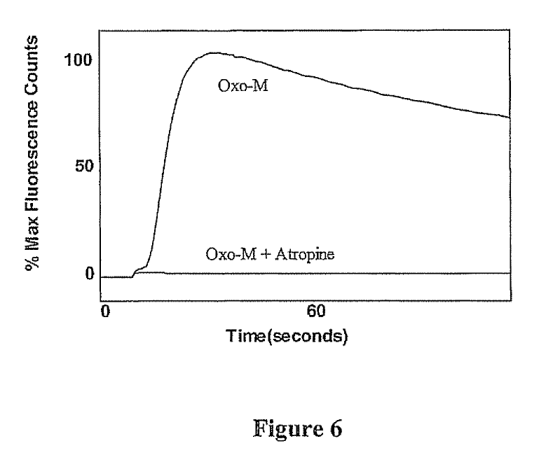

The ability to detect agonists and antagonists of the muscarinic acetylcholine receptor via its activation of the SK2 K⁺ channel using the thallium influx technique is shown in FIG. 6. A stable baseline fluorescence was observed in cells in the presence or absence of the acetylcholine receptor antagonist, atropine. Upon the addition of oxo-M, an increase in BTC fluorescence was observed. This increase in fluorescence was absent without the addition of oxo-M. Furthermore, the oxo-M stimulated increase in BTC fluorescence was totally prevented by the presence atropine. These data clearly demonstrate the ability of the thallium efflux technique to detect agonists and antagonists of the G-protein coupled muscarinic acetylcholine receptor via its activation of the SK2 K⁺ channel.

### EXAMPLE VIII

Example VIII demonstrates the applicability of the thallium influx assay to high throughput screening.

To rapidly screen for modulators that display selectivity for the ion channel, to be examined, a 384 well FLIPR was used. The instrument can simultaneously, optically, measure changes in the fluorescence of the cells in each well of a 384 well microtest plate (FIG. 5).

A voltage-gated K⁺ channel was screened for both opener and blocker compounds using conditions similar to those described above for KCNQ2 in Example IV. Screening was accomplished by a single person using a Molecular Devices FLIPR 384 equipped with a stacker at a rate of ˜48,000 samples/8 hrs.

Blocker and opener compounds identified by the thallium flux assay were validated by a two-electrode voltage clamp using the same voltage-gated channel expressed in Xenopus oocytes (Barnard, E. A., et al., Proc. R. Soc. Lond., 1982, B215, 241-246; Krafte, D., Lester, H. A., 1989, J. Neurosci. Meth., 26, 211-215). The validation rate was >80% for opener and >80% for blockers. Both the high rate of sample testing/person and the fidelity of the thallium flux assay in identifying bona fide openers and blockers of the voltage-gated K⁺ channel screened make apparent the utility of this assay for efficiently discovering molecules that can modulate the activity of cation channels.

Taken together, these examples clearly show that the methods of the invention are capable of detecting modulators of both ligand and voltage-gated K⁺ channels, as well as non-selective cation channels, in a microtiter plate format useful for high throughput screening.

What is claimed is:

1. A method for detecting and measuring the activity of ion channels, channel-linked receptors, or ion transporters expressed in cells comprising: (a) contacting cells expressing ion channels, ion channels and channel-linked receptors, or ion transporters with a signal-generating thallium sensitive agent; (b) contacting said cells with a candidate ion channel, channel-linked receptor, or ion transporter modulator; (c) contacting the cells with an assay buffer containing a thallium salt solution; and (d) detecting and measuring the signal generated by the signal-generating thallium sensitive agent to determine the effect of the candidate ion channel, channel-linked receptor or ion transporter modulator on the activity of said ion channels, channel-linked receptors, or ion transporters.

2. The method of claim 1, wherein said ion channels comprise cation channels that are permeable to thallium ions.

3. The method of claim 2, wherein said cation channels are selected from the group consisting of potassium ion channels, sodium ion channels, and calcium ion channels.

4. The method of claim 2, wherein said cation channels are potassium ion channels.

5. The method of claim 4, wherein said potassium ion channels are calcium-activated and voltage-gated channels.

6. The method of claim 4, wherein said potassium ion channels are selected from the group consisting SK channels, Maxi-K, HERG and KCNQ channels.

7. The method of claim 2, wherein said cation channels are ligand-gated VR1 channels.

8. The method of claim 2, wherein said cation channels are non-selective ion channels.

9. The method of claim 8, wherein said non-selective ion channels are selected from the group consisting of acetylcholine receptors, glutamate receptors such as AMPA, kainate, and NMDA receptors, 5-hydroxytryptamine-gated receptor-channels, ATP-gated (P2X) receptor-channels, nicotinic acetylcholine-gated receptor-channels, vanilloid receptors, ryanodine receptor-channels, IP₃ receptor-channels, cation channels activated in situ by intracellular cAMP, and cation channels activated in situ by intracellular cGMP.

10. The method of claim 1, wherein said thallium salt solution comprises a water soluble thallium salt.

11. The method of claim 10, wherein said thallium salts are selected from the group consisting of Tl₂SO₄, Tl₂CO₃, TlCl, TlOH, TlNO₃ and TlOAc.

12. The method of claim 11, wherein said thallium salt is Tl₂SO₄.

13. The method of claim 1 wherein said assay buffer is Cl⁻-free.

14. The method of claim 13, wherein the said assay buffer further comprises sodium gluconate; potassium gluconate; calcium gluconate; magnesium gluconate; HEPES and glucose.

15. The method of claim 1, wherein said cells are grown in a low Cl⁻ cell growth medium, containing no more than 2 mM Cl⁻.

16. The method of claim 15, wherein the low Cl⁻ cell growth medium comprises sodium gluconate; potassium gluconate; MgSO₄.7H₂O; NaHCO₃; calcium gluconate; NaH₂PO₄; HEPES; Glucose; 100XVitamins; 50X amino acids; and glutamine.

17. The method of claim 1, wherein said thallium sensitive agent is a thallium sensitive fluorescent agent or thallium sensitive non-fluorescent agent.

18. The method of claim 17, wherein said thallium sensitive fluorescent agent is selected from the group consisting of ANTS, Fluo-4, Fluo-3, PBFI, Phen Green, Magnesium Green, APTRA-BTC, Fluo-4FF, FluoZin-1, FluoZin-2, Mag-Fura Red and BTC.

19. The method of claim 1, wherein said thallium sensitive agent is a thallium sensitive non-fluorescent agent.

20. The method of claim 19, wherein said thallium sensitive non-fluorescent agent is selected from the group consisting of chloride, bromide and iodide.

21. The method of claim 1, wherein said channel-linked receptors are selected from the group consisting of GPCR, metabotropic glutamate receptors, muscarinic acetylcholine receptors, dopamine receptors, and serotonin receptors.

22. The method of claim 1, wherein said ion transporters are selected from the group consisting of dopamine ion transporters, glutamate ion transporters, seratonin ion transporters, sodium-potassium ATPases, proton-potassium ATPases, sodium/calcium exchangers, and potassium-chloride ion co-transporters.

23. The method of claim 1 further comprising contacting the cells with extracellular fluorescent quenching compounds after said step of contacting cells with a signal generating thallium sensitive fluorescent agent.

24. The method of claim 1, wherein said candidate modulating compounds activate or inhibit said ion channels, channel-linked receptors, or ion transporters.

25. The method of claim 1, further comprising adding a stimulus solution to the thallium salt solution.

26. The method of claim 25, wherein the stimulus solution contains agents selected from the group consisting of ionophores, KCl, nicotine, acetylcholine, muscarin, and carbamylcholine.

27. A method for identifying a modulator of an ion channel, channel-linked receptor or ion transporter comprising: (a) contacting cells expressing ion channels, ion channels and channel-linked receptors, or ion transporters with a signal-generating thallium sensitive agent; (b) contacting said cells with a candidate modulator; (c) contacting the cells with an assay buffer containing a thallium salt solution; and (d) detecting and measuring the signal generated by the signal generating thallium sensitive agent, wherein the signal generated by the signal generating thallium sensitive agent is an indication of the effect of the modulator on the activity of said ion channels, channel-linked receptors, or ion transporters.

28. The method of claim 27, further comprising the step of measuring the signal generated by the signal generating thallium sensitive agent after step (b).

29. The method of claim 27, wherein said modulator activates or inhibits said ion channels, channel-linked receptors, or ion transporters.

30. A novel Cl⁻-free assay buffer for use in thallium sensitive assays.

31. The composition of claim 29, wherein the said assay buffer further comprises sodium gluconate; potassium gluconate; calcium gluconate; magnesium gluconate; HEPES and glucose.

32. A low Cl⁻ cell growth medium containing no more than 2 mM Cl⁻.

33. The composition of claim 31, wherein the low Cl⁻ cell growth medium comprises sodium gluconate; potassium gluconate; MgSO₄.7H₂O; NaHCO₃; calcium gluconate; NaH₂PO₄; HEPES; Glucose; 100X Vitamins; 50X amino acids; and glutamine.
